(19) United States
(12) Patent Application Publication
(10) Pub. No.: US 2009/0060997 A1
(43) Pub. Date: Mar. 5, 2009

(54) PROCESS FOR PRODUCING A PHARMACEUTICAL PREPARATION FOR THERAPEUTIC TREATMENT OF ENDOMETRIOSIS CONTAINING A COMBINATION OF A GESTAGEN AND (6S)-5-METHYLTETRAHYDROFOLATE

(76) Inventors: Christian Seitz, Zeuthen (DE); Annemarie Wasserfall, Berlin (DE); Holger Zimmermann, Falkensee (DE); Konstanze Diefenbach, Berlin (DE); Kristina King, Berlin (DE)

Correspondence Address: Striker, Striker & Stenby, 103 East Neck Road, Huntington, NY, 11743, US

(21) Appl. No.: 12/185,456

(22) Filed: Aug. 4, 2008

Related U.S. Application Data
(60) Provisional application No. 60/968,168, filed on Aug. 27, 2007.

(51) Int. Cl. A61K 9/36; A61K 31/519; A61K 31/56; A61P 15/00

(52) U.S. Cl. 424/479; 514/249; 514/171

(57) ABSTRACT

A combination of an anti-androgenic gestagen at a daily dose of from an ovulation-inhibiting dose up to at most twice the ovulation-inhibiting dose and from 0.1 to 10 mg of (6S)-5-methyltetrahydrofolate are used to produce a pharmaceutical preparation for therapeutically treating endometriosis while simultaneously reducing therapy side effects including the negative effect on bone density and/or bone metabolism, reducing the risk of osteoporosis and, in the event of pregnancy, reducing the risk of congenital malformations, such as medullary tube defects, cleft lip, cleft jaw, or cleft palate, and the risk of pregnancy complications, such as detachment of the placenta and premature birth. The preparation is suitable for long-term administration, which continues daily for at least 169 days to at least two years.

### CROSS-REFERENCE

The invention described and claimed herein below is also described in U.S. Provisional Patent Application, Ser. No. 60/968,168, filed on Aug. 27, 2007. In accordance with 35 U.S.C. 119(e) the claims appended herein below should be accorded the benefit of priority of invention based on the disclosures in the aforesaid US Provisional Patent Application.

### BACKGROUND OF THE INVENTION

1. The Field of the Invention

The invention relates to the use of gestagens in combination with (6S)-5-methyltetrahydrofolate, wherein the daily dose of the gestagen amounts to a maximum of twice the ovulation-inhibiting dose, to produce pharmaceutical preparations for therapy of endometriosis with simultaneous reduction of therapy side effects, such as the negative effect on bone density/bone metabolism, the risk of osteoporosis and, in the event of pregnancy, the reduction of the risk of congenital malformations, such as medullary tube defects, cleft lip, cleft jaw, or cleft palate, and pregnancy complications, such as the detachment of the placenta and premature birth. The invention is suitable for long-term use.

2. Description of the Related Art

Endometriosis is a chronic, gynecological illness appearing primarily in 5-20% of women of childbearing age. In the technical literature, endometriosis is defined as the appearance of the endometrium or of endometrium-like tissue outside the uterine cavity. Typical symptoms of endometriosis are dysmenorrhea, dyspareunia, and pain during bowel movement. Endometriosis patients often complain about pain in the pelvic region. Hypogastric pain appearing in the second half of the cycle followed by painful menstruation and subsequent absence of symptoms until the middle of the following cycle, are often suggestive of endometriosis, but persisting pain is not rare. At any rate, about 30-40% of persons affected by endometriosis have no symptoms. The illness is then detected only by chance in connection with other diagnostic measures. In about 50-60% of the women, the diagnosis “endometriosis illness” is made by chance in the interpretation of sterility.

It is known from the technical and patent literature to treat endometriosis with drugs such as Danazol, a derivative of 17α-ethinyl-testosterone, GnRH agonists, gestagen/estrogen combinations or single-gestagen preparations.

U.S. Pat. No. 6,569,845 contains a description of a treatment of angiogenic disorders with dienogest, i.e. 17α-cyano-methyl-17-β-hydroxy-estra-4,9-dien-3-one, at a daily dose of 0.5 to 10 mg. Similar pharmaceutical preparations given as examples and which could be used widely also for the treatment of endometriosis have a dienogest content of 400 mg to 2 g.

Moore, C. et al., in “The Treatment of Endometriosis”, Drugs of Today 1999, 35 (Suppl. C): 41-52, reported results of clinical studies of the efficacy of dienogest in the treatment of endometriosis in comparison with the therapeutic regime of Danazol or of GnRH agonists. The persons affected with endometriosis received 2 mg of dienogest daily for 24 weeks. The result of the treatment was comparable to that with Danazol or with a GnRH agonist. Up to 90% of the affected persons reported irregular bleeding, but none of them reported unbearable bleeding. The efficacy of the standard treatment with Danazol is reduced by significant androgenic effects, whereas GnRH agonists are associated with “menopausal symptoms”.

Schweppe, K. W., states, in “The Position of Gestagens”, Zentralbl. Gynakol. 2003, 125: 276-280 that during continuous oral treatment with gestagens (for example with medroxy-progesterone acetate, dienogest, dydrogesterone, lynestrenol at a daily dose of 5 mg to 20 mg, referred to as low dosage and classified as an effective treatment principle of endometriosis-induced symptoms) low estrogen levels are observed. Spotting and intermediate bleeding occur frequently. This compels one to increase the dose and/or to add estrogen. Long-term relapse rates exceed 50%.

A 2005 safety data sheet for a medroxyprogesterone acetate product indicates that the single-gestagen preparations can have a negative effect on bone density, particularly if used for a long time. In combination with an estrogen, however, they can have a positive effect on the bone metabolism.

Another safety data sheet, NDA 21-584, FDA 03.22.2005, for DEPO-SUBQ PROVERA 104® (medroxyprogesterone acetate i.m.—104 mg/0.65 mL) points out that women using this preparation suffer a drop in bone mineral density, which increases with the duration of the use of this preparation and is no longer completely reversible.

Knauthe, R. and Habenicht U. F., in “Levonorgestrel has Beneficial Effects”, Exp. Clin. Endocrinol. Diabetes 106 (1998), Suppl. 1: 37 already pointed out in 1998 that the partial androgenic action of a gestagen (levonorgestrel) and not the gestagenic activity is responsible for this positive effect on the bone metabolism. Kuhl, H., in “Klimakterium, Post-menopause und Hormonsubstitution” [Climacterium, Post-menopause and Hormone Replacement], 3rd ed. Bremen, UNI-MED, 2006, 117, also stresses that certain gestagens become effective via their partial androgenic activity. Kuhl also states that androgens markedly enhance the positive effects of estrogens on bone density.

In the technical literature, osteoporosis is described as a bone degradation that cannot be reversed and that is accompanied by increased bone fragility. In Germany, more than 5 million people suffer from osteoporosis. Women are affected more often than men. Osteoporosis is a pain-free insidious phenomenon during which not only the amount of bony substance decreases, but the “architecture” of the bone changes so that it no longer withstands normal loads. A frequent consequence of osteoporosis is a fracture of the neck, of the femur, or the extremely painful fracture of the body of a vertebra. Despite treatment, the risk of further fractures is very high. The basic medication for osteoporosis is the combination of calcium with vitamin D. Drugs of first choice for osteoporosis therapy are the biphosphonates alendronate and risedronate and the selective estrogen receptor modulator, Raloxifene.

Bröll, H. et al. state in “Consensus Statement: Therapy of Postmenopausal Osteoporosis”, J. Miner. Stoffwechs. January 2007, 45-48, that the most frequent reasons for discontinuing oral therapy with biphosphonates are the side effects. The side effects of the indicated biphosphonates include abdominal pain, cold-like syndrome, constipation, peripheral edema, tinnitus and bronchitis. It is also disadvantageous that when taking a biphosphonate, because of the danger of mucous membrane inflammation, one must strictly follow the prescription, namely take the drug in the morning, on an empty stomach, with a large glass of water and while in the standing position. After ingesting the drug, one should lie down for at least one-half hour.

According to the literature, the drawbacks of Raloxifene are possible hot flashes, calf cramps, and edema.

Estrogen-containing preparations are used in hormone replacement therapy to maintain the bony mass. Estrogen-containing preparations also present some risks. They should be used for prevention of cancer of the uterine mucous membrane only in women with an intact uterus.

It is also known that endometriosis is associated with sub-fertility and is often diagnosed in the course of interpretation of an unsatisfied desire for a child. Hence, one goal of endometriosis therapy is often also to increase the probability of pregnancy.

Moreover, it is known that an insufficient folate level in pregnancy can lead to congenital malformations, for example congenital cardiac defects, congenital malformations of the urinary tract, acute lymphoblastic leukemia, cleft lip, cleft jaw, or cleft palate, or malformations of the central nervous system, such as medullary tube defects (spinal bifida or anencephaly).

The German Society for Nutrition, a registered organization, therefore recommends as a basic daily dose 400 μg of folic acid, for pregnant women 600 μg and for nursing mothers 600 μg. This is a general statement. Deficiencies of vitamin B₁₂ and folate result in identical changes in the blood picture. The folate deficiency can be compensated for by administration of folate/folic acid, but the vitamin B₁₂ deficiency is not mentioned. For this reason, the risk of a masked vitamin B₁₂ deficiency exists.

Folic acid, also known as pteroylmonoglutamic acid, N-(4-{[(2-amino-1,4-dihydro-4-keto-6-pteridinyl)methyl]amino}benzoyl)glutamic acid (empirical formula C₁₉H₁₉N₇O₆) or as folinic acid is a heat-sensitive and light-sensitive water-soluble vitamin of the vitamin B complex (vitamin B₉).

It is known that folates are present in foods predominantly as pteroyl polyglutamates. After food ingestion, these compounds are first hydrolyzed to pteroyl monoglutamates in the mucosal cells and are then absorbed in the intestines mainly by active transport.

In the liver, the predominantly non-methylated folates are converted into methylated folates, mainly as 5-methyltetrahydrofolate (5-MTHF) bound to albumin and a-macro-globulin, and further transported to the cells where they are taken up, demethylated and converted to the polyglutamate form.

The amino acid homocysteine and an enzyme requiring vitamin B₁₂ as the coenzyme take part in the demethylation.

It is also known that folate losses from foods can be caused by food preparation (cooking) and by storage. In addition, it is known that exposure of the human skin to intense UV radiation reduces the level of folic acid in the body. Persons with light skin are thereby particularly affected.

An insufficient supply of folate and/or vitamin B₁₂ hinders the homocysteine metabolism so that as a result the homocysteine concentration in the blood can increase. The concentration of homocysteine in the blood can therefore be used as an indicator of the folate content. According to Malinow, M. R. et al, in Homocyst(e)ine, Diet and Cardiovascular Disease, Statement for Healthcare Professionals from the Nutrition Committee, American Heart Association, Circulation 99, 178-182, 1999, the state of hyper-homocysteinemia is defined by the following concentration in the plasma: 16-30 μmol/L (moderate); 31-100 μmol/L (medium); >100 μmol/L (severe). A concentration, which is greater than 10 μmol/L, is considered to be critical and one above 12 μmol/L requires treatment.

An increase in homocysteine concentration, however, can also be brought about by enzyme defects and not only by a folate and vitamin B₁₂ deficiency. The correlation between elevated homo-cysteine concentration in the blood and blood vessel disorders, for example as a risk factor for cardiovascular diseases, has been under discussion for some time.

Also under discussion is the question whether folic acid/folate because of its importance for the methylation of DNA and for the stability of the DNA strand can protect from malignant diseases.

European patent EP 0 898 965 claims the use of (6S)-5-methyl-tetrahydrofolic acid or the pharmaceutically compatible salts thereof for the prevention of medullary tube defects.

European patent EP 1 044 975 discloses crystalline salts of 5-methyl-(6R,S)—, -(6S)— and (6R)-tetrahydrofolic acid and the use thereof as constituents of food supplements.

### SUMMARY OF THE INVENTION

It is an object of the present invention to provide a way of protecting endometriosis patients from a risk of osteoporosis, to reduce the endometriosis and to attain one goal of endometriosis reduction, namely the onset of pregnancy, without side effects.

It is a further object of the present invention to provide a process for producing a pharmaceutical preparation, which contains a combination of a gestagen and (6S)-5-methyl-tetrahydrofolate (METAFOLIN®) and which provides an effective therapeutic treatment of endometriosis, but which reduces therapy side effects, such as the negative effect on bone density/bone metabolism, and the risk of congenital malformations and pregnancy complications in the event of pregnancy.

These objects and others, which will be made more apparent herein after, are attained by a process of producing a pharmaceutical preparation for therapeutic treatment of endometriosis with simultaneous reduction of therapy side effects including a negative effect on bone density and/or bone metabolism, reduction of the risk of osteoporosis, and reduction of the risk of congenital malformations and pregnancy complications in the event of pregnancy, said process comprising using a combination of a gestagen and (6S)-5-methyl-tetrahydrofolate (METAFOLIN®), and wherein the daily dose of the gestagen is at most twice an ovulation-inhibiting dose and at least equal to the ovulation-inhibiting dose of the gestagen, together with or separate from one or more pharmaceutically acceptable excipients and/or carriers.

Surprisingly, we have found that the reduction of endometriosis is not accompanied by a negative effect on bone metabolism so that no reduction/drop in bone density is observed, there is no risk of osteoporosis and the risks of congenital malformations, such as medullary tube defects and cleft lip, cleft jaw, or cleft palate, and pregnancy complications such as placental detachment and premature birth in the event of pregnancy can also be reduced.

At the same time, surprisingly, by administration of the pharmaceutical preparation, it is possible to keep the known side effects of conventional drugs for treating endometriosis, for example hot flashes and changes in the lipid profile, within tolerable limits.

According to the invention, the gestagens with anti-androgenic action used in the pharmaceutical preparation for treating endometriosis are dienogest (17α-cyano-methyl-17-β-hydroxy-estra-4,9-dien-3-one), cyproterone acetate, and/or chlormadinone acetate.

The daily dose of the gestagens should amount at the most to twice the ovulation-inhibiting dose.

The daily dose of dienogest amounts to 1 mg up to a maximum of 2 mg. According to the invention, cyproterone acetate or chlormadinone acetate are also used at a daily dose of up to twice the ovulation-inhibiting dose. The ovulation-inhibiting dose of cyproterone acetate is 1 mg and that of chlormadinone acetate is 1.7 mg.

According to the invention, the objects are also attained by using from 0.1 to 10 mg of the (6S)-5-methyltetrahydrofolate in the daily dose, preferably 0.4 mg up to 1 mg of the (6S)-5-methyltetra-hydrofolate and, most preferably, 451 μg of (6S)-5-methyltetra-hydrofolate, e.g. in the form of the calcium salt of (6S)-5-methyltetra-hydrofolic acid.

The use according to the invention also makes it possible to produce a pharmaceutical preparation for continuous administration of the dosage form for at least 169 days or 25 weeks to several years, preferably for more than 2 years, and is thus, surprisingly suitable for long-term use.

Tablets, capsules, sugar-coated tablets, wafers, transdermal therapy systems, ampoules, suppositories, gels, ointments, implants, vaginal rings, or nasal sprays are suitable for the use according to the invention. The daily dose of gestagen released by the non-oral forms of the pharmaceutical preparation, such as transdermal therapy systems, ampoules, suppositories, gels, ointments, implants, vaginal rings, or nasal sprays is equivalent to once or twice the ovulation-inhibiting daily dose unit released by the oral forms or at the most equivalent to the efficacy of 2 mg of dienogest.

Moreover, the objective of the invention is reached by a process for producing a pharmaceutical preparation for the therapy of endometriosis with simultaneous reduction of therapy side effects including the negative effect on bone density/bone metabolism, of the risk of osteoporosis, as well as, in the event of pregnancy, of the risk of congenital malformations and pregnancy complications, such as medullary tube defects, cleft lip, cleft jaw, or cleft palate, detachment of the placenta and premature birth, which contains a combination of at least one gestagen and (6S)-5-methyltetrahydrofolate, together with or separate from one or more pharmaceutically acceptable excipients/carriers, wherein the daily dose of the gestagen is equal to at least the ovulation-inhibiting dose and up to at the most twice the ovulation-inhibiting dose.

Other embodiments of the pharmaceutical preparation produced by the process according to the invention are claimed in the appended dependent claims.

### PRACTICAL EXAMPLES

### Example 1

Tablets having the following composition were prepared:

Core:

| | |
|---|---|
| Dienogest | 2.000 mg |
| METAFOLIN ® | 0.451 mg |
| Lactose monohydrate | 46.349 mg |
| Microcrystalline cellulose | 24.800 mg |
| Hydroxypropylcellulose | 1.600 mg |
| Croscarmellose | 3.200 mg |
| Magnesium stearate | 1.600 mg |

All substances were appropriately mixed together, tabletted and optionally coated.

### Example 2

Tablets of the following composition were prepared:

| | |
|---|---|
| Dienogest, micronized, | 2.000 mg |
| min. 99% ≦ 20 μm, 100% < 30 μm | |
| Lactose monohydrate | 62.800 mg |
| Microcrystalline cellulose | 18.000 mg |
| Potato starch | 36.000 mg |
| Povidone K25 | 8.100 mg |
| Magnesium stearate | 1.350 mg |
| Talc | 4.050 mg |
| Crospovidone | 2.700 mg |

Dienogest micronized to an average particle size of 20 μm was mixed with lactose monohydrate, microcrystalline cellulose and potato starch. Povidone K25 was sprayed in during the granulation. After drying and addition of talc, crospovidone and magnesium stearate, the mixture of the substances was compressed into tablets having a diameter of 7 mm and weighing 135 mg.

451 μg of (6S)-5-methyltetrahydrofolate was processed separately or together with one or more pharmaceutically acceptable excipients/carriers by known methods and administered at the same time as the dienogest formulation.

### Example 3

In a clinical study, 252 women with laparoscopically diagnosed endometriosis were treated over a period of 6 months either with the GnRH agonist leuprorelin acetate (LA), 3.75 mg s.c., every 4 weeks or with 2 mg/day orally of the gestagen, dienogest (DNG). 128 patients were randomly assigned to the LA group and 124 patients to the DNG group. The efficacy of the therapy was examined, among other ways, with the aid of a pain scale (Visual Analog Scale, VAS) questionnaire that was filled out by the patient. At the end of the treatment, both of the two groups being examined showed a similar reduction in pain compared to the beginning of the study (−47.5 mm for DNG; −46.0 mm for LA). Statistical analysis showed that DNG was not inferior to LA. At the same time, it was demonstrated in a subpopulation that in the dienogest group there was no reduction in bone density whereas in the LA group the bone density dropped by 4%.

While the invention has been illustrated and described as embodied in a process of producing a pharmaceutical preparation for therapeutic treatment of endometriosis containing a combination of a gestagen and (6S)-5-methyltetra-hydrofolate, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of the prior art, fairly constitute essential characteristics of the generic or specific aspects of this invention.

What is claimed is new and is set forth in the following appended claims.

We claim:

1. A process of producing a pharmaceutical preparation for therapeutic treatment of endometriosis with simultaneous reduction of therapy side effects including a negative effect on bone density and/or bone metabolism, of the risk of osteoporosis, and of the risk of congenital malformations and pregnancy complications in the event of pregnancy, said process comprising using a combination of a gestagen and (6S)-5-methyltetrahydrofolate and wherein a daily dose of said gestagen is from an ovulation-inhibiting dose to twice said ovulation-inhibiting dose.

2. The process as defined in claim 1, wherein the congenital malformations and the pregnancy complications are medullary tube defects, cleft lip, cleft jaw, cleft palate, detachment of the placenta and premature birth.

3. The process as defined in claim 1, wherein said gestagen is dienogest, cyproterone acetate, or chlormadinone acetate.

4. The process as defined in claim 1, wherein said daily dose comprises from 1 to 2 mg of dienogest, an equivalent amount of cyproterone acetate, or an equivalent amount of chlormadinone acetate.

5. The process as defined in claim 1, wherein a daily dose of said (6S)-5-methyltetrahydrofolate in the preparation is from 0.1 to 10 mg.

6. The process as defined in claim 5, wherein said daily dose of said (6S)-5-methyltetrahydrofolate is from 0.4 to 1 mg.

7. The process as defined in claim 1, wherein a daily dose of said (6S)-5-methyltetrahydrofolate in the preparation consists of 451 μg of a calcium salt of said (6S)-5-methyltetrahydrofolic acid.

8. The process as defined in claim 1, wherein said preparing comprises preparing a number of daily dose units containing said gestagen and said (6S)-5-methyltetrahydrofolic acid for continuous administration of said combination over a period of from at least 169 days or from 25 weeks up to at least two years.

9. The process as defined in claim 1, wherein the pharmaceutical preparation is in the form of tablets, capsules, sugar-coated tablets, wafers, transdermal therapy systems, ampoules, suppositories, gels, ointments, implants, vaginal rings, or nasal sprays.
